(19) United States
(12) Patent Application Publication
(10) Pub. No.: US 2009/0030456 A1
(43) Pub. Date: Jan. 29, 2009

(54) Leather furniture

(76) Inventor: Ming-Yang Kuo, Ta-Chia Chen (TW)

Correspondence Address: MCCRACKEN & FRANK LLP, 311 S. WACKER DRIVE, SUITE 2500, CHICAGO, IL, 60606, US

(21) Appl. No.: 11/881,332

(22) Filed: Jul. 25, 2007

(51) Int. Cl. A61F 5/00

(52) U.S. Cl. 606/237; 5/622

(57) ABSTRACT

A leather furniture includes: a frame; a leather sheet wrapped around the frame; and a connecting unit including a first connecting element and a second connecting element. The first connecting element has a first secured part secured to the frame, and a first engaging part extending from the first secured part. The second connecting element has a second secured part secured to the leather sheet, and a second engaging part extending from the second secured part. The first engaging part releasably engages the second engaging part in a snap engaging manner.

### BACKGROUND OF THE INVENTION

1. Field of the Invention

This invention relates to a leather furniture, more particularly to a leather furniture having a connecting unit for connecting a leather sheet to a frame in a snap engaging manner.

2. Description of the Related Art

As shown in FIG. 1, a conventional leather furniture 10, such as a massage bed, includes a bed frame 11, a panel 14 supported on the bed frame 11, a leather sheet 13 wrapped around the bed frame 11 and the panel 14, and legs 12. When the bed frame 11 is made from a wood material, nailing or sewing method can be employed for connecting the leather sheet 13 to the bed frame 11 (the example shown in FIG. 2 uses the nailing method). However, when the bed frame 11 is made from a metal material, the aforesaid fixing methods cannot be used. In addition, the leather sheet 13 fixed by the nailing or sewing method is not completely attached to the bed frame 11, i.e., parts of the leather sheet 13 between nails 114 are not tightly attached to the bed frame 11. Moreover, the aforesaid fixing methods are laborious.

Therefore, there is a need in the art to provide a leather furniture that includes a connecting unit for tightly and easily connecting a leather sheet to a frame.

### SUMMARY OF THE INVENTION

Therefore, the object of the present invention is to provide a leather furniture that can overcome the aforesaid drawbacks of the prior art.

According to this invention, a leather furniture includes a frame, a leather sheet wrapped around the frame, and a connecting unit including a first connecting element and a second connecting element. The first connecting element has a first secured part secured to the frame, and a first engaging part extending from the first secured part. The second connecting element has a second secured part secured to the leather sheet, and a second engaging part extending from the second secured part. The first engaging part releasably engages the second engaging part in a snap engaging manner.

### BRIEF DESCRIPTION OF THE DRAWINGS

Other features and advantages of the present invention will become apparent in the following detailed description of the preferred embodiments of this invention, with reference to the accompanying drawings, in which:

FIG. 1 is a perspective view of a conventional leather furniture;

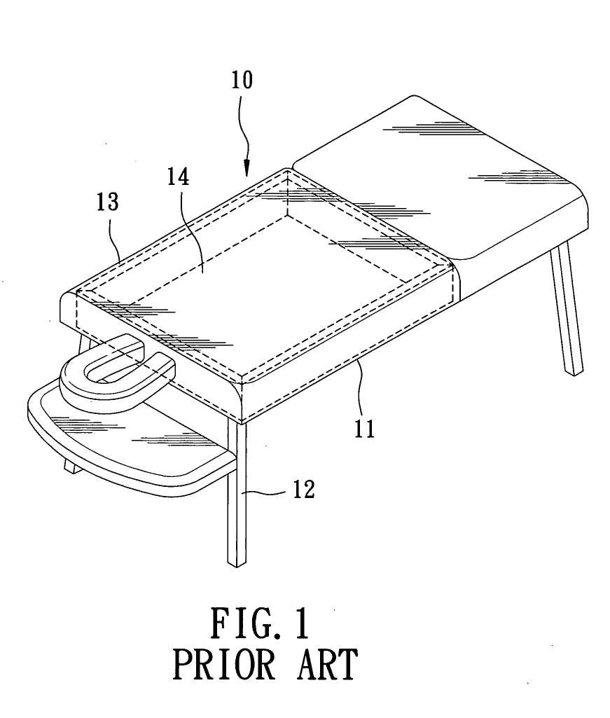

FIG. 2 is a fragmentary perspective view of the leather furniture shown in FIG. 1;

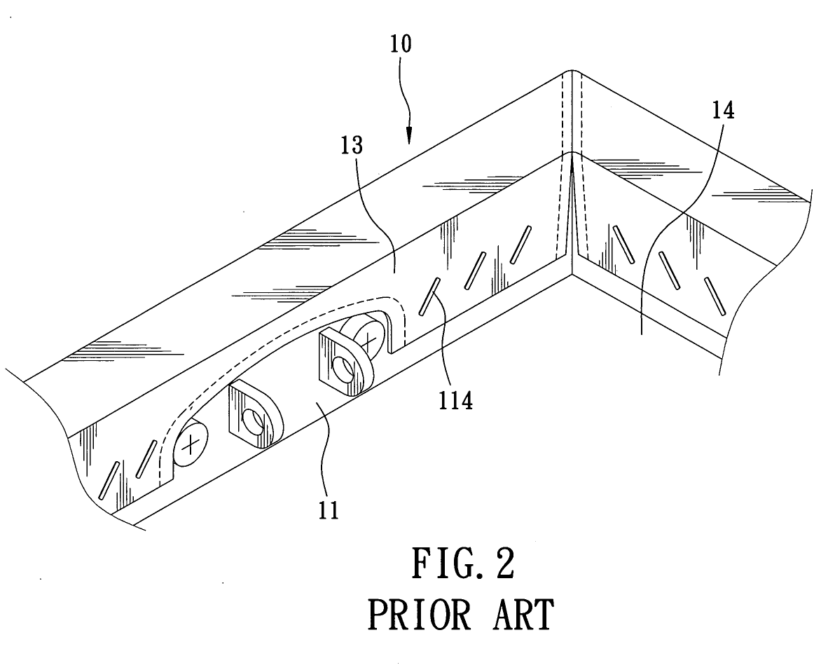

FIG. 3 is a fragmentary perspective bottom view of the first preferred embodiment of a leather furniture according to this invention at an engaging state;

FIG. 4 is a fragmentary perspective bottom view of the first preferred embodiment of the leather furniture according to this invention at a disengaging state;

FIG. 5 is across-sectional view of the first preferred embodiment at the engaging state; and

FIG. 6 is across-sectional view of the second preferred embodiment of the leather furniture according to this invention at an engaging state.

### DETAILED DESCRIPTION OF THE PREFERRED EMBODIMENTS

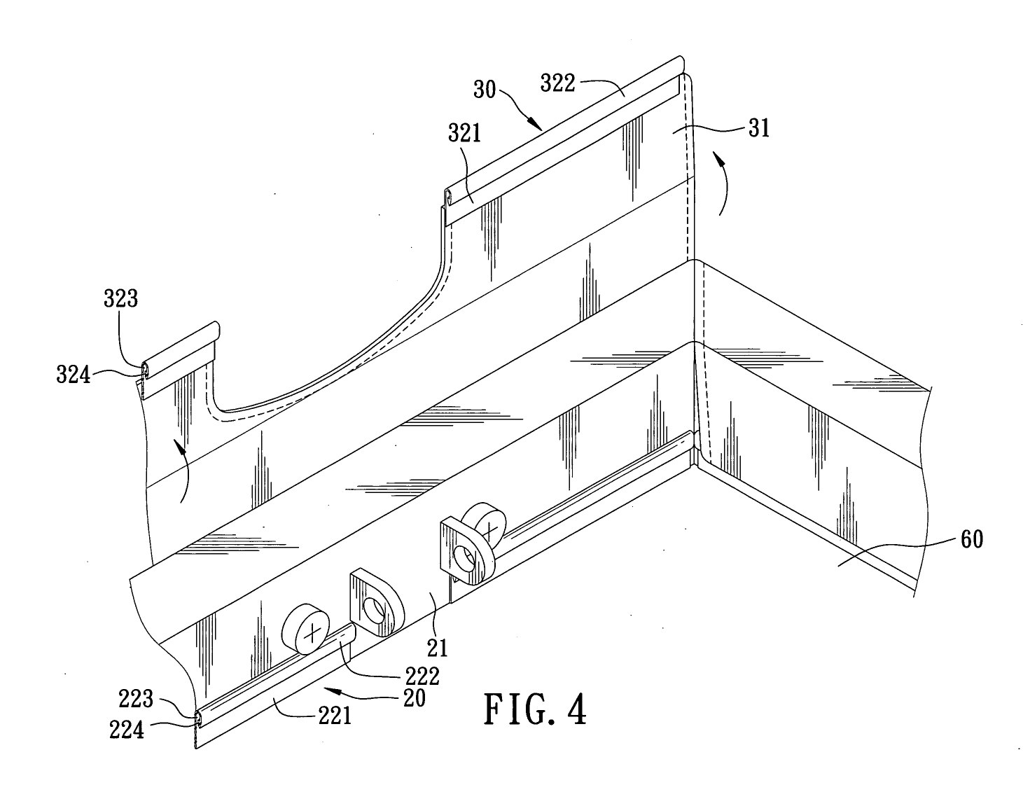

Referring to FIG. 4, the first preferred embodiment of a leather furniture according to the present invention is shown to be in the form of a bed that includes a bed frame 21, a leather sheet 31 wrapped around the bed frame 21, and a connecting unit including a first connecting element 20 and a second connecting element 30. The first connecting element 20 has a first secured part 221 secured to a lateral edge of the bed frame 21, and a first engaging part 222 extending and bent by 180° from the first secured part 221 so as to define a first gap 223 between the first secured part 221 and the first engaging part 222. The first engaging part 222 of the first connecting element 20 further includes a neck portion 224.

The second connecting element 30 has a second secured part 321 secured to the leather sheet 31, and a second engaging part 322 extending and bent by 180° from the second secured part 321 so as to define a second gap 323 between the second secured part 321 and the second engaging part 322. The second engaging part 322 of the second connecting element 30 further includes a shoulder portion 324.

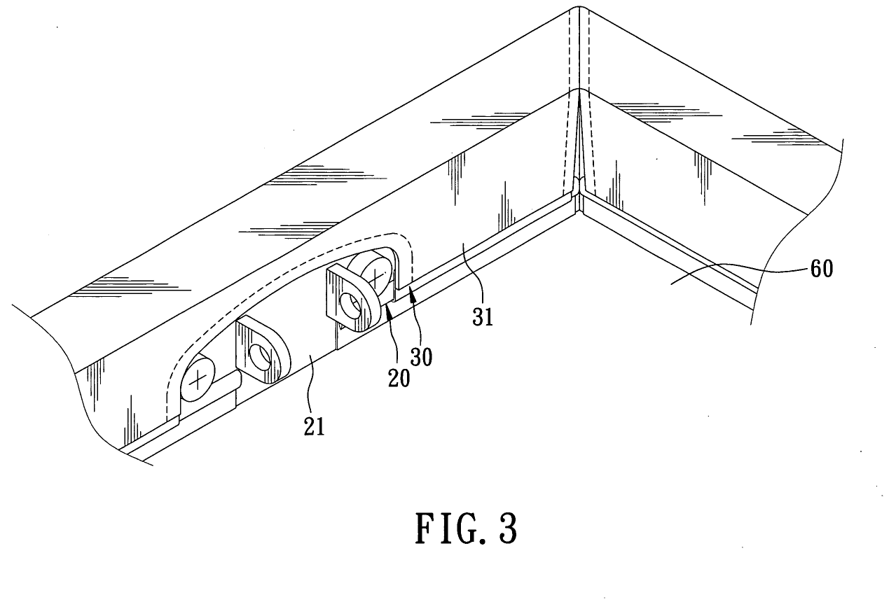

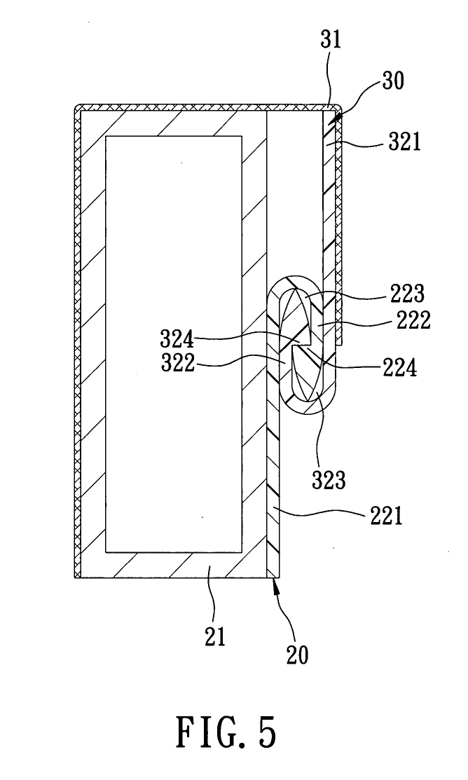

As shown in FIGS. 3 and 5, the first engaging part 222 of the first connecting element 20 releasably engages the second engaging part 322 of the second connecting element 30 in a snap engaging manner. Specifically, for fixing the leather sheet 31 to the bed frame 21, the second connecting element 30 secured on the leather sheet 31 is tightly drawn toward the first connecting element 20 secured on the bed frame 21, and the shoulder portion 324 of the second engaging part 322 is pushed into the first gap 223 of the first connecting element 20. Thus, the neck portion 224 of the first engaging part 222 and the shoulder portion 324 of the second engaging part 322 releasably engage each other, i.e., at an engaging state (see FIG. 3) so as to achieve the fixing purpose. In the meantime, the neck portion 224 of the first engaging part 222 is positioned in the second gap 323 of the second connecting element 30. The first engaging part 222 has a tapered end portion that defines the neck portion 224 and that has a maximum width greater than that of an open end of the second gap 323. The second engaging part 322 has a tapered end portion that defines the shoulder portion 324 and that has a maximum width greater than that of an open end of the first gap 223. Note that the first and second engaging parts 222, 322 are slightly resilient so as to permit insertion of the neck portion 224 and the shoulder portion 324 into the second gap 323 and the first gap 223, respectively.

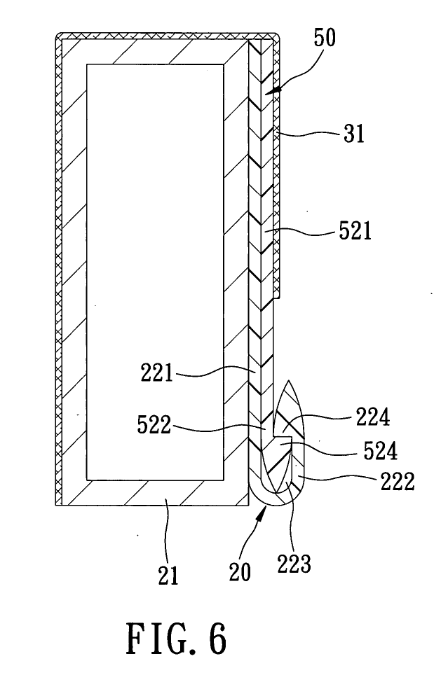

FIG. 6 illustrates the second preferred embodiment of the leather furniture according to this invention. In this preferred embodiment, the connecting unit is similar to that of the first preferred embodiment except for the structure of the second connecting unit 50.

In detail, as shown in FIG. 6, the second connecting element 50 has a second secured part 521 secured to the leather sheet 31, and a second engaging part 522 having a shoulder portion 524 and directly extending from the second secured part 521 so that no gap is defined between the second secured part 521 and the second engaging part 522.

Preferably, in this invention, the leather furniture further includes a panel 60 mounted on the bed frame 21. An upper side of the panel 60 is wrapped by the leather sheet 31.

The leather furniture may be a leather chair in other embodiments of this invention.

According to the present invention, the leather sheet 31 can be firmly and easily fixed to the bed frame 21 through snap engaging connection via the connecting unit, and assembling time and costs for fixing the leather sheet 31 to the bed frame 21 are reduced.

While the present invention has been described in connection with what are considered the most practical and preferred embodiments, it is understood that this invention is not limited to the disclosed embodiments but is intended to cover various arrangements included within the spirit and scope of the broadest interpretation and equivalent arrangements.

What is claimed is:

1. A leather furniture comprising:
a frame;
a leather sheet wrapped around said frame; and
a connecting unit including a first connecting element and a second connecting element, said first connecting element having a first secured part secured to said frame, and a first engaging part extending from said first secured part, said second connecting element having a second secured part secured to said leather sheet, and a second engaging part extending from said second secured part, said first engaging part releasably engaging said second engaging part in a snap engaging manner.

2. The leather furniture of claim 1, wherein said first engaging part of said first connecting element includes a neck portion, said second engaging part of said second connecting element including a shoulder portion, said neck portion of said first engaging part releasably engaging said shoulder portion of said second engaging part.

3. The leather furniture of claim 2, wherein said first connecting element defines a first gap formed between said first secured part and said first engaging part, said shoulder portion of said second engaging part extending into said first gap when said neck portion of said first engaging part engages said shoulder portion of said second engaging part.

4. The leather furniture of claim 3, wherein said second connecting element defines a second gap formed between said second secured part and said second engaging part, said neck portion of said first engaging part extending into said second gap when said neck portion of said first engaging part engages said shoulder portion of said second engaging part.
